(19) United States
(12) Patent Application Publication
(10) Pub. No.: US 2021/0290152 A1
(43) Pub. Date: Sep. 23, 2021

(54) WOUND ASSESSMENT, TREATMENT, AND REPORTING SYSTEMS, DEVICES, AND METHODS

(71) Applicant: DermaGenesis LLC, Pompano Beach, FL (US)

(72) Inventor: Richard Vogel, Pompano Beach, FL (US)

(21) Appl. No.: 16/823,567

(22) Filed: Mar. 19, 2020

(51) Int. Cl. A61B 5/00; G16H 10/60; G16H 15/00; G06N 3/08; G06T 17/00

(52) U.S. Cl. CPC A61B 5/445; A61B 5/7264; G16H 10/60; G06T 2210/41; G06N 3/08; G06T 17/005; G16H 15/00

(57) ABSTRACT

Exemplary devices, systems, and methods provide wound treatment plans, healing predictions, and processing of medical supplies customized for the wounds of individual patients as the wounds progress through the healing process.

### FIELD OF THE INVENTION

The invention generally relates to the field of wound healing therapy and, more particularly to assessing and monitoring wound healing, determining and updating wound treatment plans, and improving reporting for reimbursement claims. Medical treatment of a patient with a wound typically calls for assessment of wound sizes, repeated over time to provide an indication of the patient's progress.

### BACKGROUND

Treating open wounds (e.g., surgical wounds, traumatic wounds, burns, venous ulcers, diabetic ulcers, arterial ulcers and decubitis ulcers) that are too large and/or infected to spontaneously close has long been a troublesome area of medical practice. Healthcare costs for wound care in the US alone are estimated in tens of billions of dollars annually.

The wound healing process is a dynamic pathway optimally leading to restoration of tissue integrity and function. Healing pathways are set into motion at the moment of wounding, and require the successive, coordinated function of a variety of cells and the close regulation of degradative and regenerative steps, including coagulation, inflammation, ground substance and matrix synthesis, angiogenesis, fibroplasia, epithelialization, wound contraction, and remodeling.

The wound healing process applies to both acute and chronic wounds. However, in chronic wounds, the sequential process of wound healing has been disrupted leading to the interruption of the normal, controlled inflammatory phase or cellular proliferative phase. Many factors can contribute to poor wound healing. The most common include local causes such as wound infection; tissue hypoxia; repeated trauma; the presence of debris and necrotic tissue; and systemic causes such as diabetes mellitus, malnutrition, immunodeficiency, and the use of certain medications. Wound infection is a particularly common reason for poor wound healing. While all wounds are contaminated with bacteria, whether a wound becomes infected is ultimately determined by the host's immune competence, the type of wound-pathogen(s) present, the formation of a microbial biofilm, and/or the numbers of bacteria present.

Aside from infection, a variety of other factors can influence healing of wounds. These include excessive exudate, necrotic tissue, poor tissue handling, and impaired tissue perfusion, as well as clinical conditions such as advanced age, diabetes, and steroid administration.

Chronic wounds are prone to excess exudate and the formation of necrotic tissue, which in turn supports the growth of microbes. Initial debridement of necrotic tissue is important for wound bed preparation, so that wound treatment can progress.

Because wound treatment can be costly in both materials and professional care time, a treatment that is based on an accurate assessment of the wound and the wound healing process can be essential. There are a few wound parameters that may assist a clinician in determining healing progress of a wound. For example, wound area and volume measurements may provide a clinician with knowledge as to whether or not a wound is healing and, if the wound is healing, how rapidly the wound is healing. Wound assessment is important to properly treating a wound since improper or incomplete assessment may result in a wide variety of complications. Infections at a tissue site that go untreated may result in permanent damage or even death to a patient.

Generally wound measurement technologies for many years incorporated an object, or objects, physically placed onto a patient near the wound. An example of a commercially available wound measuring device is sold by McKesson, 5×7 inch, in a form of a disposable clear plastic sheet with a circular, bull's eye grid marked in centimeters and inches, that is placed atop a patient's wound. However, needing to physically place something onto a patient has at least the inherent disadvantage from the issue of sterility of any object being placed onto or near the patient's wound. Further, when a plastic sheet, marker object, etc. is placed atop or near the wound, the used object must undergo proper disposal. Also, placing measuring devices or marker objects atop, or near, a patient wound can be associated with patient discomfort or pain. Consequently, there remain unmet needs for improvements in wound measurement and assessment technology.

A clinician often examines wound tissue for its color, texture, and size to determine how a wound is healing. Wound measurement is an important parameter for determining the progress of wound healing. Wound tissue includes a wound bed and periwound areas or wound edges. Health of a wound and certain problems in a wound may be detected from the color of wound tissue. For example, normal granulation tissue has a beefy, red, shiny textured appearance and bleeds readily, whereas necrotic tissue (i.e., dead tissue) may either be yellow-gray and soft, generally known as “slough” tissue, or hard and black/brown in color, generally known as “eschar” tissue. A clinician may observe and monitor these and other wound tissues to determine wound healing progress of the overall wound and specific wound regions. However, these observations, without more downstream integration and precise tracking of wound metrics including size and volume, are underutilized in predicting recovery, adjusting recovery plans, and processing reimbursement claims.

Moreover, knowledge and skill levels of healthcare workers involved in assessing and treating wounds varies widely, geographically as well as within healthcare systems generally. Doctors, nurses, technicians, home care providers and others may be involved in assessing and/or treating a wound, but the various backgrounds and levels of expertise (or lack of expertise) of each respective healthcare worker means a great number of patients with wounds receive inferior treatment than if the best expertise available among select highly experienced doctors were always brought to bear. A need exists to provide more uniform availability and applicability of such expertise in wound treatment among healthcare workers of all different backgrounds and skill levels.

### SUMMARY

An exemplary method of patient customized wound treatment comprises storing a digital record of a wound containing 3D wound scan data, the 3D wound scan data being produced by scanning the wound while manipulating a 3D camera around a center of the wound; collecting responses from a medical professional to predetermined questions pertaining to wound treatment; automatically selecting one or more specific medical products or treatments for use treating the wound, the selection being based on the digital record and collected responses; and transmitting a signal to one more users identifying the specific medical products or treatments. Such a method may further comprise scanning the wound with a 3D camera; manipulating the 3D camera around a center of the wound during the scanning step; and producing a 3D model of the wound from imaging data of the 3D camera, the 3D model being displayed or displayable on a screen and manipulatable on the screen to show a back or underside of the wound. A further step may be performed involving fulfilling an order of medical supplies that includes the specific medical products or treatments in a treatment plan.

An exemplary method of monitoring wound debridement comprises scanning a patient's wound in need of debridement, the scan being performed with a 3D camera system to produce a first digital record; debriding the patient's wound after the first digital record is produced; scanning the patient's wound within a predetermined period of time after the debridement, the scan producing a second digital record; determining metrics for quantitative comparison of the patient's wound as recorded in the first digital record versus in the second digital record; and transmitting a report containing the first and second digital records and the quantitative comparison to a medical health professional. Such a method may further comprise changing or updating a wound healing plan for the patient's wound based on the transmitted report. The transmitted signal may be or at least include a medical reimbursement claim (such as but not limited to an insurance reimbursement claim).

Another exemplary method of patient customized wound treatment comprises storing a first wound healing plan that includes a first timeline of treatment for a patient wound; receiving digital records of the patient wound on a recurring basis over a duration of time required by a patient wound to make progress healing, the digital records including wound scan data; assessing, on the recurring basis, deviation from the first wound healing plan based on the digital records; and changing or updating the first wound healing plan to a second wound healing plan when the assessed deviation exceeds a threshold, wherein the second wound healing plan includes a second timeline of treatment for the patient wound different from the first timeline of treatment.

Exemplary devices and programs for executing such methods as described above are also disclosed.

### BRIEF DESCRIPTION OF THE DRAWINGS

FIG. 1A is a depiction of a medical healthcare professional scanning the wound of a patient.

FIG. 1B is a networked system of devices and users which may transmit, receive, or exchange data with exemplary devices according to embodiments herein disclosed.

FIG. 2A is an exemplary device for wound scanning and generation of customized wound treatment plans.

FIG. 2B is an exemplary wound scanning device.

FIG. 2C is the device of FIG. 2A separated into two respective elements.

FIG. 3 is a method for patient customized wound treatment.

FIG. 4 is an interface providing a list of digital records of wound scans for a plurality of patients.

FIG. 5 is an interface with a home screen menu for reviewing 3D wound scan data and visuals for a particular patient.

FIG. 6 is a wound identification interface.

FIG. 7 is a wound debridement interface.

FIG. 8 is a wound scan interface.

FIG. 9 is a wound scan orientation and wound boundary definition interface.

FIG. 10 is a wound measurement preview interface.

FIG. 11A is a wound boundary editing interface.

FIG. 11B is the wound boundary editing interface of FIG. 11A after changes have been made by a user.

FIG. 12 is an interface with an upload progress popup bar.

FIG. 13 is a wound drainage interface.

FIG. 14 is a wound filler interface.

FIG. 15 is a wound characteristics interface.

FIG. 16 is the wound characteristics interface showing another aspect.

FIG. 17 is a treatment plan interface.

FIG. 18 is the patient home screen interface.

FIG. 19 is an exemplary method for creating/editing patient records with wound scans, detections, and measures.

FIG. 20 is an exemplary method of wound scanning, detection, and measurement.

FIG. 21 is an exemplary method of wound detection.

FIG. 22 is an exemplary method of wound measurement.

FIG. 23 is an exemplary method of determining a wound volume.

FIG. 24 is a method for sharp debridement of a wound.

FIG. 25 is an interface for debridement reports.

FIG. 26 is a method of patient customized wound treatment and healing prediction.

FIG. 27 is an interface showing expected wound healing progress.

FIG. 28 is the expected healing progress interface with a drop down menu opened to different prediction presets.

FIG. 29 is an actual wound healing progress interface.

### DETAILED DESCRIPTION

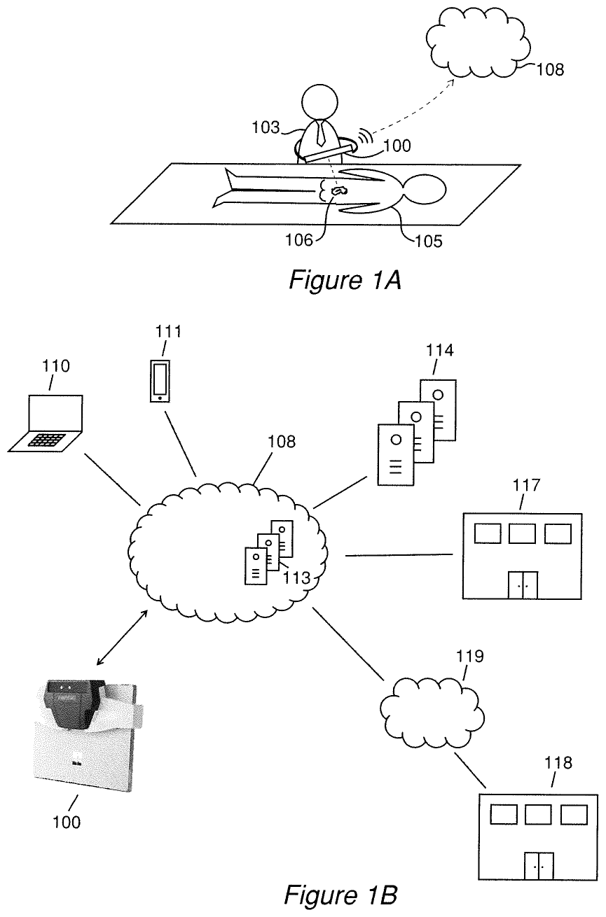

FIG. 1A shows a user 103 using a device 100 for wound healing therapy. The device 100 is handheld and may be a system of physically separate (or at least separable) independent devices working together. The device 100 is handheld by the user 103 for easy manipulation in proximity to a patient 105 and a wound 106 of the patient 105. At a minimum the device 100 includes a 3D camera (a term which, though singular, may refer to a coordinated collection of sensors some of which may individually be referred to as cameras and collectively may sometimes be referred to as a module). At least some 3D camera hardware suitable for use with some embodiments is available commercially from manufacturers and suppliers such as Intel® (Real Sense 3-D camera), Orbbec® (Astra 3-D camera), and Stereolabs® (ZED stereo 3-D camera). The device 100 also includes a display and means for user input, e.g., touchscreen integral with the display. Data collected by the device 100 from the wound 106 and from the user 103 may be collected and stored locally on the device 100. In exemplary embodiments, at least some or all of the data collected by the device 100 is transmitted over one or more networks for subsequent uses. In FIG. 1A the device 100 transmits information wirelessly to a network 108 generally depicted by a cloud symbol.

The term “user” may be used herein to mean any clinician, medical or healthcare professional (e.g., doctor, nurse, technician, assisted living staff, caretaker), family member of a patient, patient, artificial intelligence entity, or other person or device who/that interacts or interfaces with a method, device, or system according to the invention.

FIG. 1B expands upon the interconnectedness of network 108 with a variety of other devices, networks, and systems which provide a wide range of practical uses and application of the data originating from the patient wound 106, user 103, and device 100. Device 100 generally sends and receives information over the network 108. The information generally includes, for example, 3D wound scan data and the results of one or more medical questionnaires completed by a user 103. Exemplary uses of the wound information include three-dimensional (3D) modeling of the wound for improved visualization by healthcare professionals, automated or streamlined processing of reimbursement claims related to wound related healthcare, automated or semi-automated treatment recommendations or treatment plans, and automated or semi-automated ordering and fulfillment of orders of medical supplies necessary for individualized treatment of wounds.

FIG. 1B shows devices 110 and 111 which may be but are not limited to personal computers, laptops, smartphones, phones, tablets, phablets, displays, smartwatches, other wearables, and other devices. These devices may be used to view, process, and/or analyze data from the device 100 which may itself be a personal computer, laptop, smartphone, phone, tablet, phablet, smartwatch, other wearable, or other type of device. Displays on such devices may be used to view, assess, and manipulate 3D wound models produced from the device 100. Remote computers or servers 113 or 114, arranged as cloud servers and/or servers accessible over the cloud, may be used to perform computation tasks that reduce the processing demands of device 100. The servers 113 or 114 may be used for storing digital records of wound scans and related data. Companies and institutions 117 and 118 such as insurance providers may interact with information received from and sent to device 100 over network 108, sometimes directly or sometimes through means of other networks 119.

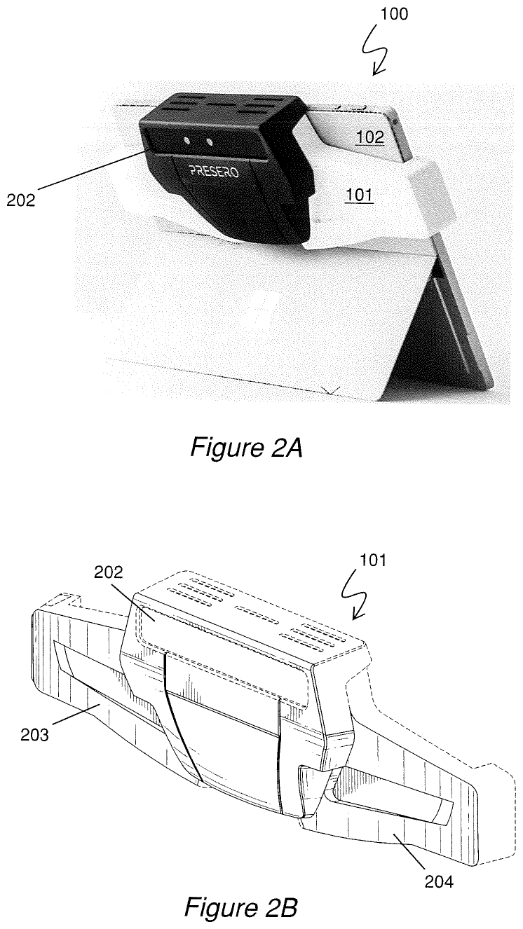

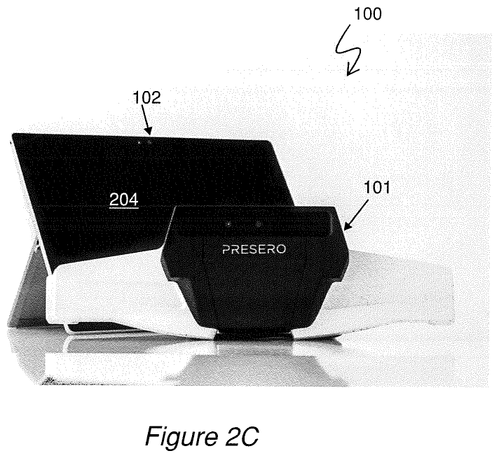

FIG. 2A shows a device 100 standing alone for ease of viewing. The device 100, exemplary for purposes of this disclosure but not intended to be limiting beyond the claims below, comprises two independent but cooperating devices. The device 100 includes an imaging device 101 and a user interactive device 102. In some embodiments, the device 100 may be a single or unitary device or piece of hardware. In this circumstance devices 101 and 102 may be subcomponents of the device 100 which are not physically distinguishable. The device 100 may be a portable mobile device such as a mobile phone (e.g., a smartphone) or a tablet.

The device 101, shown alone in FIG. 2B, includes a 3D camera situated behind a protective screen 202. The device 101 includes arms 203 and 204 for removably attaching to device 102. The device 102 may be a relatively generic (at least prior to running custom software program methods discussed in greater detail below) standalone device, such as a tablet or laptop or 2-in-1 device. The devices 101 and 102 may communicate and exchange data continuously while the device 100 is in use.

FIG. 2C shows the devices 101 and 102 separated from one another. From the depicted angle display 204 is visible. Subsequent figures, discussed below, illustrate exemplary sequences of interfaces which may be displayed and presented to a user using the devices 100/101/102. The same interfaces presented or presentable to a user on a display 204 are used to both present scanning data and 3D models to a user as well as receive user input which affects outputs of the device 100.

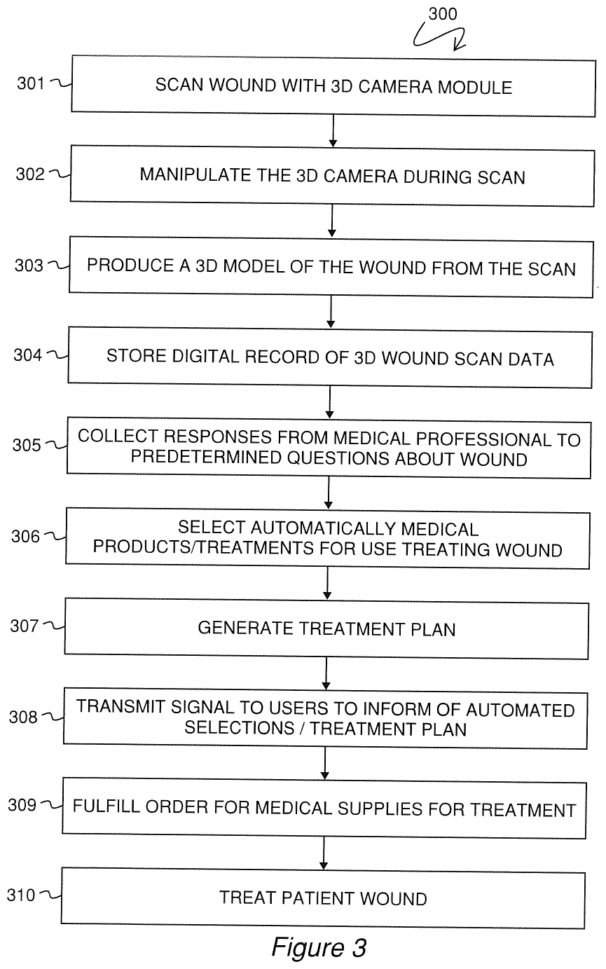

The device 100 and its components permit methods of patient customized wound treatment. FIG. 3 is a method 300 for patient customized wound treatment, offering an automated or semi-automated process for determining a treatment plan for a patient on an individualized basis. That is, the method 300 may be employed to produce and carry out a treatment plan that is unique to a particular patient, or at least unique to a particular set of wound conditions and parameters which differ for one patient's wound as compared to many other patients' wounds. While there are multiple advantages of such customization and tailoring of treatment to individual patients, arguably one of the greatest advantages is the potential to improve the extent of wound recovery and the speed with which the wound recovers, as contrasted with generic treatment plans that do not adequately account for differences among wounds of different patients, or even different wounds on the same patient. Methods disclosed herein, including but not limited to method 300, account for and yield different results based on differences of one patient's wound as contrasted with another patient's wound, or based on differences of a first wound and a second wound where the wounds belong to the same patient.

Method 300 provides an overview of steps, many of which are illustrated in greater detail through exemplary user interfaces discussed below and following in the figures listing. Briefly, method 300 generally begins at block 301 with a scan of a wound using a 3D camera. For best results the 3D camera is manipulated during the scan, block 302, in order for the camera to image the surfaces of the wound at a variety of different angles. At block 303 a 3D model of the wound is produced from the 3D wound scan data. A digital record is stored at block 304 containing the 3D wound scan data and, often, the 3D model. The precise configuration of the data and the form in which it is stored may vary among embodiments depending on the hardware (e.g., servers) and data handling protocols being used for storage. Before, during, and/or after the scan, but preferably temporally close with the scan in any event, questions are presented to the user (generally in this case, a medical professional is desired but not always necessarily required) and responses to the questions collected and recorded at block 305. The questions are generally predetermined, and answers to the questions may be limited to predetermined finite lists of options (e.g., the questions may be designed as multiple choice questions) to streamline the automated processes of the responses and limit the introduction of user error. From the 3D wound scan data combined with the collected responses, automatic selection is made at block 306 of medical products and/or treatments for use on the wound in question. The products/treatments and the timing of their use may be combined together in a generated treatment plan. A signal containing information of the selections and/or treatment plan may be sent to various users or interested parties at block 308. One possible receiving party is an order fulfillment company or service which fulfills at block 309 the order of medical supplies specified by the automated selection of block 306. Ultimately the patient, specifically the patent's wound in question, is treated at block 310.

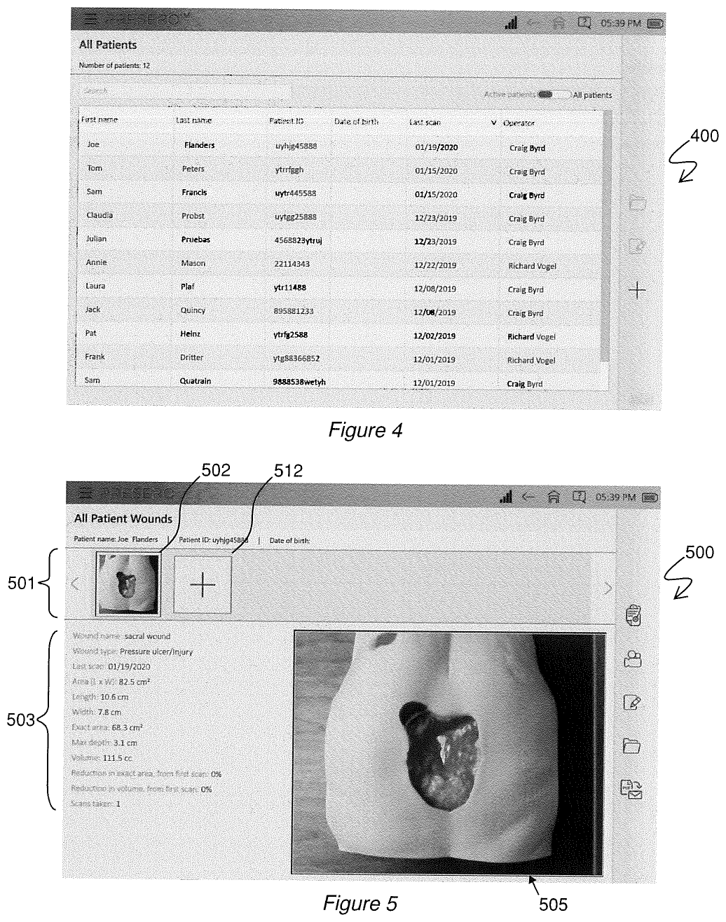

FIGS. 4 through 17 will now walk through a series of exemplary user interfaces which correspond with a majority of the steps outlined in FIG. 3. The interfaces may be displayed to a user in series, that is one interface at a time one after the other. Each interface may require the user to make one or more selections in the interface concerning a wound care topic prior to advancing to the subsequent interface. The series of interfaces may include, but are not necessarily limited to, wound type, debridement, orientation, wound border confirmation, wound drainage, wound filler, and wound characteristics. The order of such interfaces may vary among embodiments, but satisfactory completion of all interfaces remains highly desirable for complete and consistent treatment plans regardless of the user's level of expertise. Thus the ability of the user to skip an interface (or any interface) may be limited or barred without the user making a conscious selection by clicking, tapping, or otherwise giving a command or input (e.g., voice command) to the device 100 to indicate user acknowledgement of the topic presented by the interface.

FIG. 4 shows an interface 400 which provides a list of stored digital records of a plurality of wounds, which may be of different patients or of the same patient, or both. The interface 400 for providing an overview of stored patients and their scans may include presentation of different information, but in general it contains information usable to distinguish one patient from among a plurality and some information that is or is derived from wound scan data. In FIG. 4, interface 400 shows a table in which first name, last name, and unique patient IDs are presented to differentiate patients. A column for date of birth is available but with the information hidden or unpopulated. Scan data in the table of interface 400 includes a calendar date of the last time each wound (for each patient) was scanned. The table of interface 400 further includes identification of the user who operated the device 100 for capturing the most recent or up-to-date scan for each respective patient or wound. From interface 400, a user can access an existing patient record, edit an existing patient record, or add a new patient record.

FIG. 5 shows an interface 500 to which the display of device 102 changes after a patient is selected by a user in interface 400. Interface 500 is a home screen menu for an individual patient. An objective of interface 500 is to provide a clean interface limited to details essential to the development of treatment plans. A ribbon menu 501 contains thumbnail images 502 for all wounds for which scans exist (and generally for which the wound treatment is ongoing; scans of healed wounds may be archived and not included in ribbon menu 501) for a single patient. Each thumbnail is for a single wound, with a result that each thumbnail may represent one or a plurality of scans (depending on how many scans have been taken of any given wound). The patient is identified in a bar above the ribbon menu 501. The thumbnails in the ribbon menu 501 are individually selectable by a user. When a thumbnail 502 is selected, qualitative and quantitative metrics 503 derived for the particular wound and from the latest available scan for that wound are displayed in the interface 500, in this case on the left hand side. On the right hand side of the interface 500 is a visual 505 of the actual 3D wound scan data from the latest scan for the wound in question. The nature of visual 505 may be configured to be one of several different options. The visual 505 may be a two-dimensional photograph, for example. Or, as another example, the visual 505 may be a three-dimensional model of the wound derived from the 3D wound scan data. In this case the visual 505 may be rotated and angled without leaving interface 500.

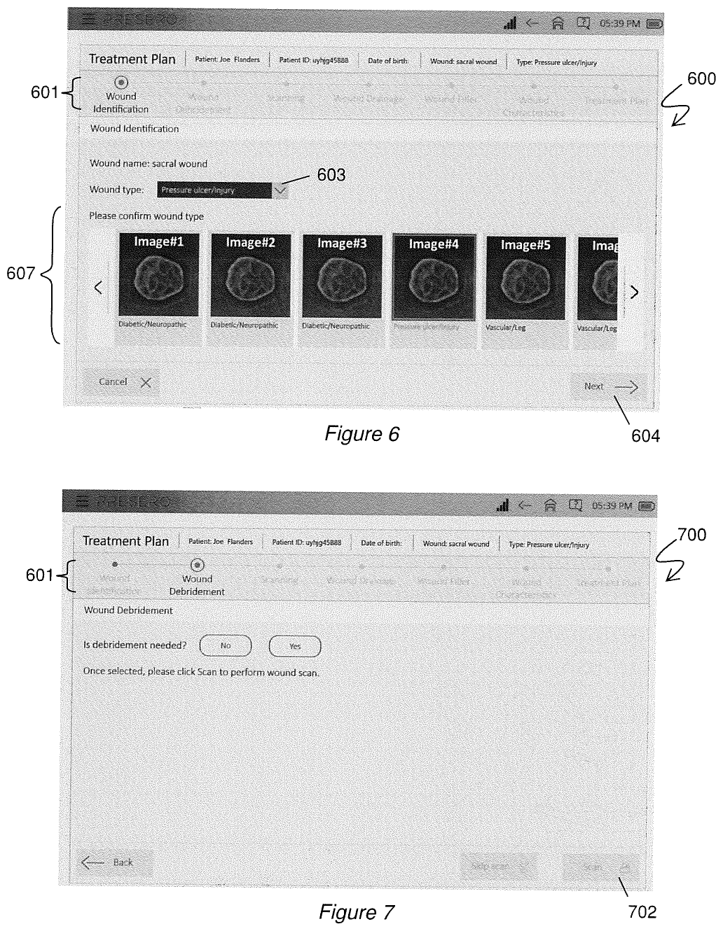

FIG. 6 shows an interface 600, the first of several which collectively lead a user step by step through a process of generating a treatment plan, including the collection of new 3D wound scan data. It should be appreciated that the production of the treatment plan may entail the selection and use of 3D wound scan data stored for variable amounts of time. The wound scan may be performed and the resulting data stored moments before the final production of a treatment plan. Alternatively, the wound scan may be performed and the resulting data stored minutes (e.g., 10 minutes or less), hours (e.g., 1, 4, 8, 12, or 24 hours or less), or days (e.g., 1, 2, or 3 days or less) before the final production of a treatment plan. Generally the time between scanning/storing and final production of a treatment plan is minimized so that the treatment plan is as tailored as possible to the patient's existing wound as it is and not how it was at some point in the past. Generally wound scan, recording, and production of a treatment plan based on the recorded wound can all occur within 24 hours or less, 12 hours or less, 8 hours or less, 6 hours or less, or 2 hours or less.

Across the top of interface 600 as well as subsequent interfaces is a progress bar 601 which identifies steps required of the user and which step the user is at (which interface is presently being displayed to the user). The steps may vary some among embodiments, but the exemplary steps shown in progress bar 601 are wound identification, wound debridement, scanning, wound drainage, wound filler, wound characteristics, and treatment plan. The majority of interface 600 is dedicated to input fields pertaining to wound identification.

Many patients, especially in older age demographics such as 70 and up or 80 and up, have multiple wounds. The presence of multiple wounds on a single patient means patient identification alone, such as name and birthdate, are insufficient as fully identifying information for the best possible patient specific and wound specific treatment plans. In interface 600, a specific, single, one wound can be identified by information such as, but not necessarily limited to, wound name (e.g., sacral wound) and wound type (e.g., pressure ulcer/injury). Wound types may include but aren't necessarily limited to: diabetic/neuropathic, pressure ulcer/injury, vascular/leg, surgical, traumatic, and atypical.

The wide range of wound types and the wide range of personnel who may be users of exemplary embodiments means a significant risk exists of user error in identifying wounds according strictly to a user's own judgement. Advantageously, to address this concern, interface 600 uses a drop-down selection 603 with a finite list of wound type options and a pictographic menu 607 that pairs exemplary images of different wound types with the names of the different wound types. A user may use interface 600 concurrently with the patient and the patient's wound present and make a side by side comparison of the patient's actual wound in the room and the exemplary pictures of different wound types within the pictographic menu 607. In some embodiments the pictographic menu 607 may be used alone, that is without the drop down selections 603. Once the selections in interface 600 have been made by a user the ‘Next’ button 604 is pressed to proceed to the next interface. Note that button 604 and similar buttons in subsequent interfaces which allow a user to advance within the series of interfaces may be non-selectable without the user first providing an input or acknowledgement in the presently displayed interface. The button 604 and similar advancement buttons on other interfaces may be greyed out until the user provides a response to the inquiry or inquiries of the interface presently displayed. After the user has provided the requested input, the advancement button color may change to no longer be greyed-out. Colors and shading other than grey may be used to indicate to the user that the advancement button is not yet available for selection.

FIG. 7 shows interface 700. Interface 700 advantageously corrects for yet another common error in the field of wound identification and imaging. For development of a treatment plan for a wound in its most up-to-date state and progress level, the presence of necrotic tissue in imaging used for production of that treatment plan can be problematic. Generally, if necrotic tissue is present, sharp debridement may be necessary prior to imaging, not after imaging, for the purpose of creating a treatment plan for further treating the wound after the imaging appointment. Failure of a user to debride the wound prior to imaging may lead to a less than optimal treatment plan being produced by the process. This error is exacerbated by the fact that imaging of a wound prior to wound sharp debridement can in fact be desirable for other contexts, at least one of which is discussed in a further embodiment below. Thus a user may encounter scenarios where sharp debridement is desirable before scanning a wound as well as scenarios where debridement is undesirable before scanning a wound. Interface 700 is a simple but significant interface that appears prior to providing the option for the user to scan a wound. Interface 700 queries the user whether (sharp) debridement is necessary, for which the user must make a selected answer, such as ‘yes’ or ‘no’. In some embodiments a user may be required to select an answer to the question before scanning can be commenced. For instance, the scan button 702 may be greyed out and unselected until one of the answer options to the debridement query has been selected. In effect interface 700 forces a user to consciously confront the question of debridement prior to performing a scan, whether or not debridement is actually performed. This step minimizes the risk of a user scanning a wound that requires sharp debridement but which has not been debrided, and then a less than optimal treatment plan resulting from the scan. As many professionals in the wound care space appreciate, debridement may come in the form of sharp debridement or chemical debridement. Generally sharp debridement involves cutting (e.g., with a scalpel) or otherwise manually removing material from a wound such as slough and dead cells. Chemical debridement on the other hand generally involves chemical treatments which may be incorporated into gauzes and other dressings left on a wound for an extended duration (e.g., 24 hours or longer). The device 100 may include recommendation for a dressing with a chemical debridement agent, e.g., as part of a treatment plan.

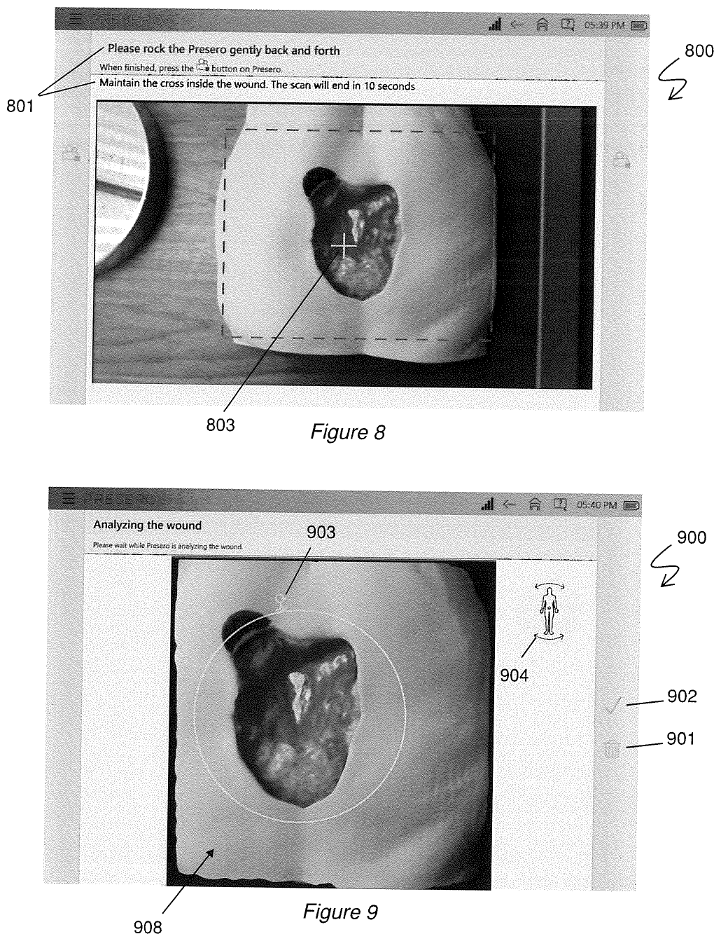

After the scan button 702 is selectable to a user and selected by the user from interface 700, a feed from the 3D camera of device 101 is fed in real time to the display 204 of device 102 as in interface 800 of FIG. 8. Accompanying display of an image feed from the 3D camera are instructions 801 for the user to follow while performing the scan. In an exemplary embodiment, the instructions 801 include instruction to rock the device 100 (e.g., back and forth) and to maintain an indicia (e.g., a cross 803) inside what the user perceives to be the outermost boundary of the wound. The instructions 801 may also include a timer which reads out the remaining time required for the scan. The instructions 801 may further indicate a user to move the device 100 closer to or further from the wound. The ideal focal length may vary among patients and wounds, depending on circumstances such as but not limited to the environmental lighting conditions, the lightness or darkness of the wound, and the wound's depth. The device may automatically factor these and other variables into account to determine an optimal focal length and correspondingly instruct the user to move nearer or further away from the wound until at the optimal distance or within an optimal range. The instruction may be as simple as a color-coded output, e.g., a green light or symbol when the device is at an acceptable focal distance and/or a red light or symbol when the device is at an unacceptable focal distance from the wound. Generally acceptable focal distances may be within the range 25 to 75 cm but may vary depending on the hardware of a specific embodiment.

The presence yet simplicity of the instructions 801 have multiple advantages. A variety of users—from doctors, to nurses, to technicians, to caretakers, and more—may readily use a device 100. Regardless of the user's level of expertise, there is minimal to no confusion from a user causing him or her to fall into the trap of incorrectly assuming the device is a point-and-click camera as opposed to correctly appreciating that it is a 3D scanner. While much of the functionality of an exemplary device 100 is substantially automated, manipulation of the device 100 within the same environment of the wound to be scanned remains a task for the user that is important to producing the best and most accurate as possible scan of a wound. The manipulation is relatively straightforward but arguably an easy oversight for some users, especially given the omnipresent familiarity of using 2D cameras on smartphones, which in complete contrast to the device 100, require a user to remain as still as possible while capturing image data (not rocking a device back and forth and around the object being imaged). Thus, the presence of the simple yet potentially counterintuitive instructions 801 concurrent with the imaging feed in interface 800 encourages accurate usage of the device by users, which in turn leads to more accurate scans and thus better treatment plans for the wound being treated.

During the scanning step, millions and sometimes billions of data points may be produced which collectively characterized a canopy of the wound. Although the word “imaging” is sometimes used in this disclosure, the word as used herein is not intended to be limited to two-dimensional images, like photographs, with which in everyday parlance “image” is often treated as a synonym.

FIG. 9 shows an interface 900 which may be presented to a user after the scanning is complete but while the device 100 is analyzing the millions or billions of data points collected. Once device 100 finishes its analysis, a user may also be given the option to discard 901 the scan and perform another scan. The interface 900 may include a depiction of the 3D model 908 constructed or being constructed from the 3D camera data and allow the user to manipulate the model to assess whether to keep 902 or discard 901 the scan used to produce the model. Tool 903 is an elliptical wound boundary aid. This tool allows a user to very generally define an area that contains a wound boundary. A user may move and/or rotate the perimeter of the tool 903 so that the wound boundary as perceived by the user is contained within the perimeter. Further refinement of the precise location and contours of the wound boundary is performed systematically by the device 100 subsequent to this request for user input. The device 100 may select a default location of the perimeter of tool 903 which a user needn't move or adjust if the user does not desire to provide such input to the system.

Interface 900 further includes a tool 904. Tool 904 together with tool 903 advantageously allow a user to orient the model with respect to overall body orientation. It is not necessary for a user to position the patient in a particular way with respect to the device 100. That is to say a user may scan a wound from any side or angle without regard for the orientation of device 100 with respect to the patient or wound, or vice versa. A user may take a scan from any orientation with respect to the patient and then correct the orientation within interface 900. As a specific example, the wound explicitly depicted in FIG. 9 is a sacral pressure ulcer. It is located near the lower back at the bottom of the spine. In the figure it is possible to make out on one side of the wound a slight depression in the skin corresponding with the lower spine and on an opposite side of the wound the start of the intergluteal cleft (natal cleft). Tool 904 includes a symbol of a body with clearly distinguishable head and feet, for example. Interface 900 allows a user, using tool 904, to orient the symbol of the body so that it matches the orientation of the scan (as in this example apparent from the position and alignment of the recognizable anatomical landmarks).

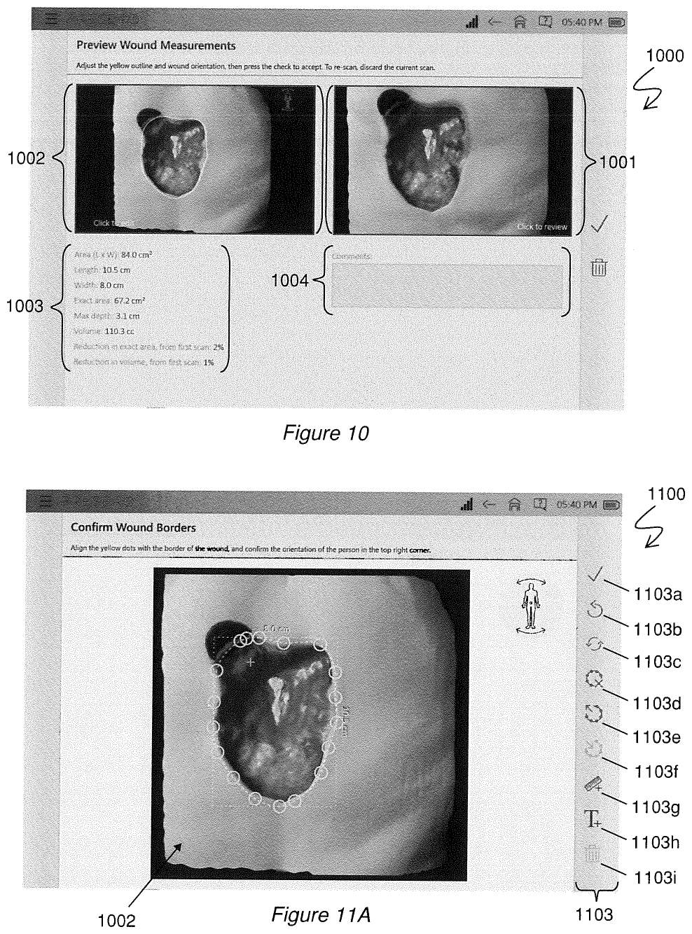

FIG. 10 shows an interface 1000 presented to a user after both scanning is complete and analysis of the scan data is complete. An objective of interface 1000 is to provide the most important metrics and elements of the wound scan record just created for the user to review. Interface 1000 includes four key elements and may contain little or nothing more than these four elements. The four elements depicted in FIG. 10 are the manipulable 3D model 1001, a 2D top plan view image 1002 showing a provisional wound boundary overlay, metrics 1003 (including size and, if applicable, changes in size from the most recent scan within the stored records available to the system), and user note input space 1004. Each of these four elements serves a distinct function, and all four together represent a core of the new scan digital record being created.

The model 1001 corresponds with the model 908 from interface 900. Clicking on the window for model 1001 within FIG. 1000 will temporarily open an interface with similar tools as in interface 900 for exploring and manipulating the 3D model for visual inspection by a user. The interface allows the user to manipulate the model for a thorough inspection from different angles or vantages. Clicking on the window for 2D image 1002 opens interface 1100, discussed below. The measurements within metrics 1003 may be limited to metrics most frequently used by healthcare professionals in the diagnosis and monitoring of wound condition, progress, and prognosis. They include and may or may not be limited to area, length, width, exact area, max depth, and volume. If multiple scans exist with which a comparison is performable, reduction in exact area (from first and/or prior scan) and reduction in volume (from first and/or prior scan) may be included. The metrics 1003 give a quick and immediate snapshot to a user of automatically produced quantitative assessment of the wound that was just scanned. Many users, especially technicians, doctors, and nurses, may have individual comments or observations that they desire to store with the wound scan records. Such comments may be entered in space 1004.

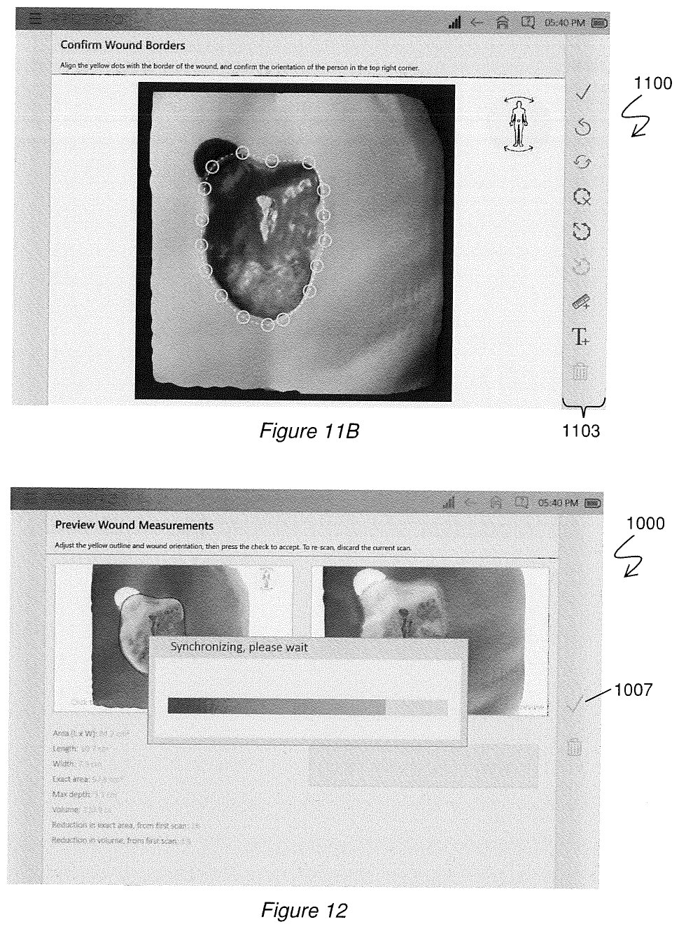

If a user clicks or selects the 2D image 1002 with the boundary overlay, the display switches to interface 1100 in FIG. 11A. Interface 1100 contains an enlarged depiction of the 2D image together with a menu 1103 of tools for manipulating the wound boundary overlay. In interface 1100 the wound boundary overlay may include a plurality of user manipulable points (which may appear as points, circles, balls, or other symbols for example) dispersed around the boundary, which in essentially all cases will form a closed shape (the beginning and end of the boundary, to the extent such terms are applicable, meet at the same point). The boundary generated automatically as disclosed elsewhere in this disclosure may comprise tens, hundreds, or thousands of points depending on the resolution of the image and other factors. Generally interface 1100 makes a small subset of boundary points overridable by a user. The menu 1103 includes tools for adding or removing points subject to user override. A user can touch and drag any of the points indicated with indicia showing that they are selectable (here, small open circles) and the boundary algorithm will adjust adjacent points to maintain the closed shape of the boundary and generally maintain other smoothing boundary parameters. Besides the options to add and remove user controlled points, menu 1103 options may include rotation of the wound boundary relative to the image, textual annotations, automated measurement annotations, and more.

FIG. 11A shows an exemplary selection of tools in menu 1103, though it will be appreciated that other embodiments may have additional, fewer, and/or other tools. Each tool is represented by a respective icon. Icon 1103a is selectable by a user to indicate satisfaction with the presently displayed boundary and the user's desire to move to the next screen. Icon 1103b is selectable by a user to return to the last selected point of the boundary. Icon 1103c is selectable by a user to return to the very first point. Icon 1103d is selectable to eliminate which user-controlled point is presently selected. Icon 1103e is selectable to expand a point outward (e.g., in a radial direction away from the wound's center). The extend to expansion may be predetermined, e.g., 10% perimeter growth per user click on the icon. Icon 1103f is the opposite. Icon 1103f is selectable to retract a point inward (e.g., in a radial direction toward the wound's center). Icon 1103g is a ruler tool. After selecting icon 1103g, a user can anchor each respective end of a virtual ruler, after which it will present on the display the real distance from the scan indicated by the user. Distances may be expressed in cm or mm or whatever predetermined unit the user desires. Icon 1103h is selectable to allow a user to add text which gets saved as part of the patient record together with the scan data. For instance, a user may add text such as “concerned about exposed bone greater than 15 cm” or the like. Icon 1103i is selectable to allow a user to eliminate any changes made since last the icon 1103a was selected or since the menu 1103 was last opened.

For purposes of illustration, FIG. 11B shows interface 1100 after a user has moved two user manipulable points of the wound boundary near the top left corner of the wound. The remainder of the boundary, especially in the vicinity of the adjusted points, are automatically adjusted by the software. Ultimately an objective of the software is to produce a highly accurate and highly precise geometry of the wound boundary for use in determining the metrics 1003. However medicine and medical treatment is and foreseeably will remain subject to the oversight of human users, especially doctors, and the interface 1100 allows users like doctors a desirable level of control and final say over the otherwise automated wound boundary determination process of exemplary embodiments. The ability to override certain determinations of the device, including the wound boundary, may be restricted to certain types of users or users of predetermined authorization (e.g., doctors may have an override option whereas homecare worker may not have an override option).

FIG. 12 shows the interface 1000 once more, but after the user has clicked or otherwise selected an icon 1007 indicating to the system that the details of blocks 1001, 1002, 1003, and 1004 are satisfactory, and that the 3D wound scan data and all other data pertaining thereto is satisfactory for storing as a digital record. At this stage the device 100 synchronizes the locally collected data to the cloud 108 and interconnected devices, as discussed above in connection with FIG. 1B. In an exemplary embodiment this synchronization with other digital records, some or all of which may generally be stored geographically remote from the user and patient, occurs within just a few minutes of the new scan being performed. The result is exceedingly fast access to the new medical records by other institutions and personnel, such as other doctors, healthcare supply suppliers, and insurance companies.

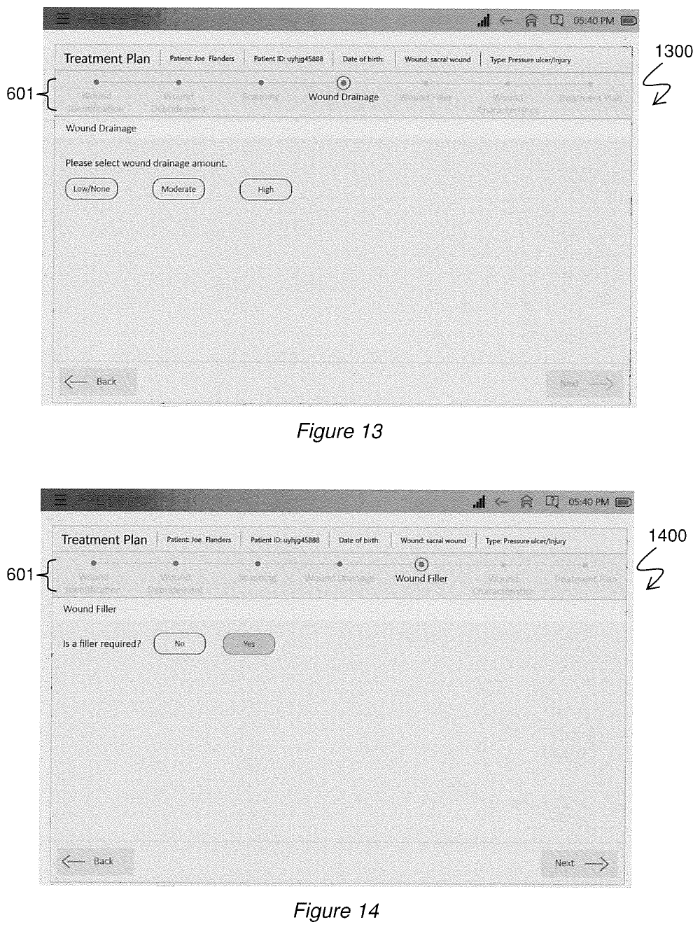

FIG. 13 is an interface 1300. Progress bar 601 shows to a user that the process leading to a production of a treatment plan is now at ‘wound drainage’. The user has at this stage completed wound identification, wound debridement (if applicable), and scanning. In interface 1300 the user is asked to select from a plurality of options an amount of wound drainage. The options shown are low/none, moderate, and high. In some embodiments, additional levels of drainage may be provided as options. However the three levels shown in FIG. 13 are advantageous as striking a balance of simplicity and specificity that minimizes having distinctions without a significant difference for purposes of producing a treatment plan and facilitates efficient response times from the user in answering the query.

FIG. 14 is an interface 1400 with the progress bar advanced to ‘wound filler’ step. Here the user is asked whether the patient wound requires filler. This may be answered simply by way of selection between a few options, e.g. just two options.

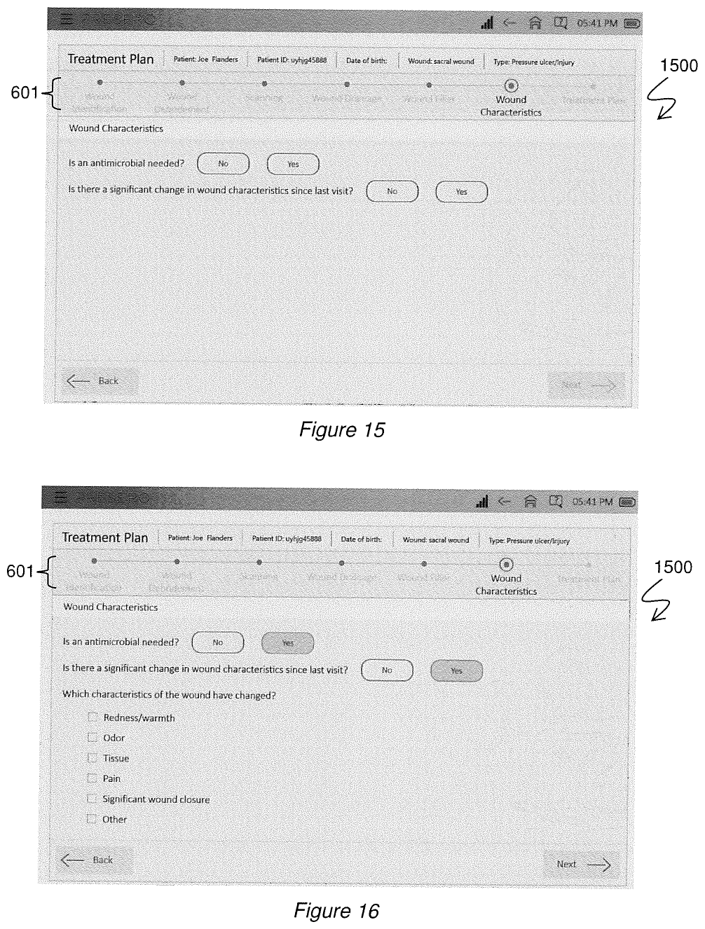

FIG. 15 is an interface 1500 with the progress bar advanced to a ‘wound characteristics’ step. Here the user is presented with questions such as whether an antimicrobial is needed, and whether there are any significant changes in wound characteristics since the last visit. The latter question exists in view of the fact that the frequent timeline several weeks to months required for full wound recovery means a vast majority of wound scans will not the be the very first scan of the wound.

FIG. 16 shows interface 1500 after a step of updating the user questionnaire as the user fills out particular questions. In this case, after user selection of ‘yes’ to the question of whether significant changes in wound characteristics are present since the last visit, a menu of characteristics is displayed from which the user is able to check off which characteristics have changed and leave unselected characteristics which have not changed. Wound characteristics which may be selected include but are not limited to redness/warmth, odor, tissue, pain, significant wound closure, and others.

The separation of questions about the patient wound through different interfaces and/or steps and levels of a single interface has been found to facilitate user compliance in answering all questions. Making most if not all questions subject to a finite number of predetermined answer options further facilitates consistent survey results which proves consequential for subsequent processing, especially automated processing. Patterns of treatment for individual patients and groups of patients are also able to be assessed based on the tailored number of survey questions and survey responses available.

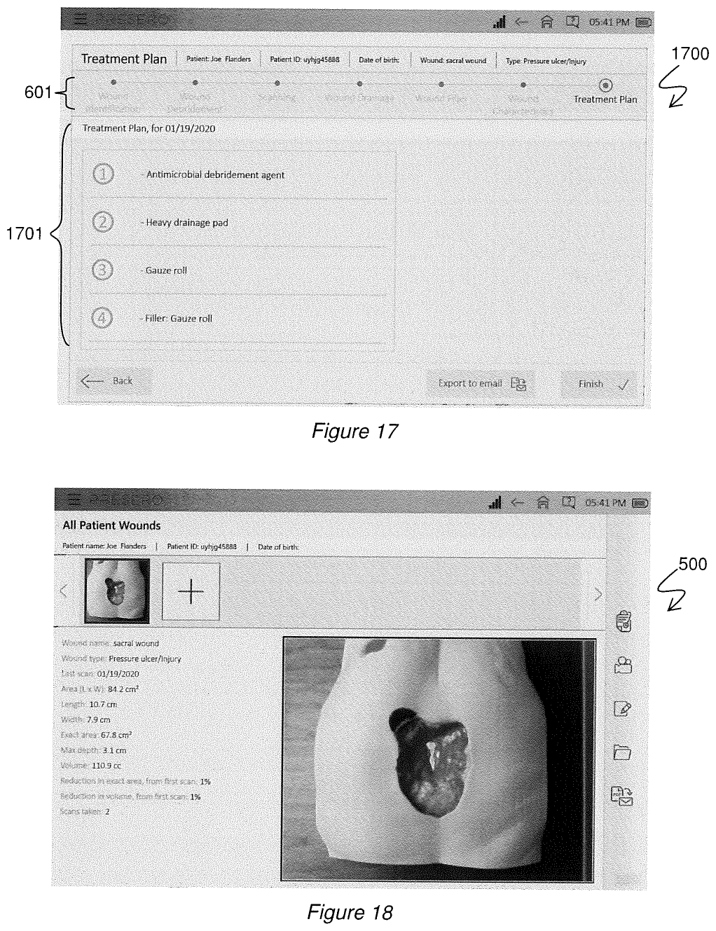

FIG. 17 shows an interface 1700 which presents a treatment plan 1701 produced from an automatic selection showing one or more specific medical products or treatments for use in treating the wound, with the selection being based on the most recent scan (sometimes in combination with data from one or more prior scans) and collected responses from the user (most recent responses, and sometimes in combination with data from responses collected in connection with one or more prior scans). An exemplary treatment plan may specify which specific medical products (e.g., dressings) should be obtained and used on the wound. A non-limiting list of examples includes antimicrobial debridement agent, heavy drainage pad, gauze roll, and a filler (which also may be gauze). An exemplary treatment plan may specify a date or dates at which to use the specific products/dressings on the wound. The wound treatment plan 1701 depicted in FIG. 17 is for a single date. An exemplary wound treatment plan may provide a sequence to the medical products and/or treatments proposed. In FIG. 17, the treatment plan 1701 provides instruction for first using an antimicrobial debridement agent, second using a heavy drainage pad, third using a gauze roll, and fourth using a gauze filler.

In some embodiments the treatment plan 1701 is generated using a decision tree which the device 100 navigates using the scan data and the series of questions presented to the user. Decisions trees may be customized, e.g., by different healthcare providers or groups of healthcare professionals personnel from which are expected to use a device 100. In this way the device 100 offers customization to reflect and accommodate subjective elements of wound therapy and healthcare generally.

The system inputs and outputs may be organized in a decision tree for which there is technically no limit on the amount of endpoints. Practically speaking, some exemplary decision trees have between 100 and 300 distinct endpoints. In alternative embodiments, neural networks or other machine learning techniques may be used to generate the treatment plan using not only scan data and questionnaire answers specific to one patient, but also based on large amounts of data for patient populations. Such large amounts of data generally exceed the ability of any human medical team to process or organize into meaningful treatment decisions.

The underlying decision tree or machine learning process to a device 100 may be amended or customized over time. For instance, a first decision tree may be determined and used for a first period of time and then adjusted to a second decision tree which is an amended version or replacement to the first decision tree. Doctors may for example amend decision trees once or twice a month or once or twice a year. Changes to decision trees may include but are not limited to changing specific treatment options (e.g., medium absorption gauze versus high absorption gauze) or the number of tree endpoints.

Irrespective of whether a decision tree or machine learning or some other alternative is used to generate the treatment plan 1701, the device 100 has the significant advantage that a relatively inexperienced healthcare professional will be able to produce for a patient the same treatment plan as that which would result from the patient's consultation with a team of wound care experts.

FIG. 18 shows a return to interface 500, the patient home screen. As compared to interface 500 depicted in FIG. 5, the quantitative metrics 503 have been updated in FIG. 18 to reflect storage of digital records of two scans instead of one scan. Comparative metrics among scans (e.g., reduction in exact area, reduction in volume) now show the progress or lack of progress in the healing of the patient's wound.

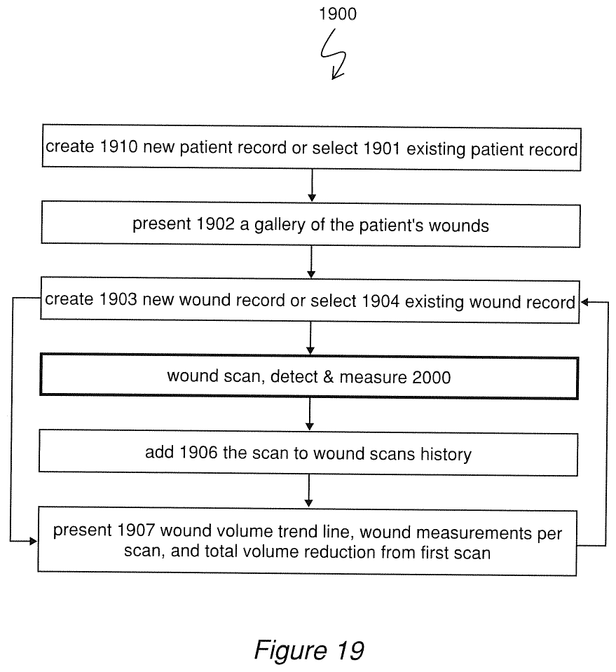

FIG. 19 is a method 1900 which may be performed using the unique user interfaces discussed above. The first block includes creating 1910 a new patient record or selecting 1901 an existing patient record. An exemplary interface for performing these steps is interface 400 of FIG. 4. After a specific record is opened, interface 500 of FIG. 5 presents 1902 a gallery of the patient's wounds. The user may add or edit wound location and type to the wound record, and/or add/edit patient details in the patient record. The user may create 1903 a new wound record by selecting/clicking the empty slot 512 for a thumbnail image, or else the user may select 1904 an existing wound record by selecting thumbnail 502 (or other thumbnails, if present). The user may then use the device/system to perform wound scanning, detection, and measurement 2000 using the series of exemplary interfaces 800, 900, 1000, and 1100. The new scan is added 1906 to wound scans history ribbon menu 501. The device/system then presents 1907 results such as but not limited to wound volume trend line, wound measurement per scan, and total volume reduction from the scan in interface 500 of FIGS. 5 and 18.

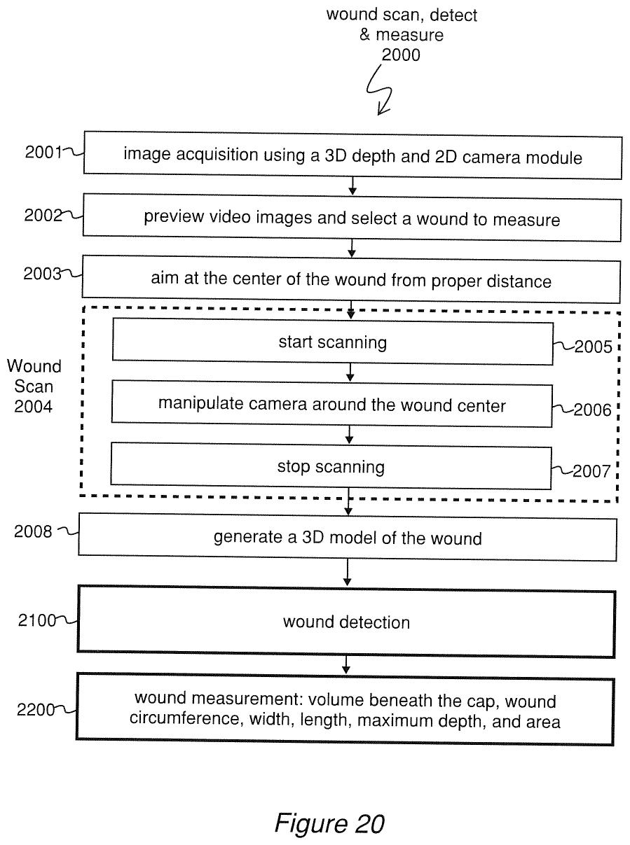

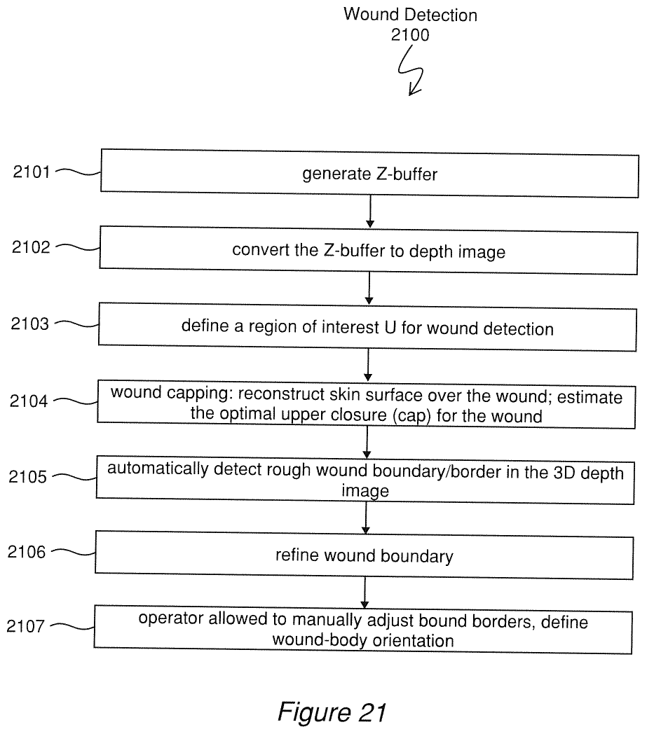

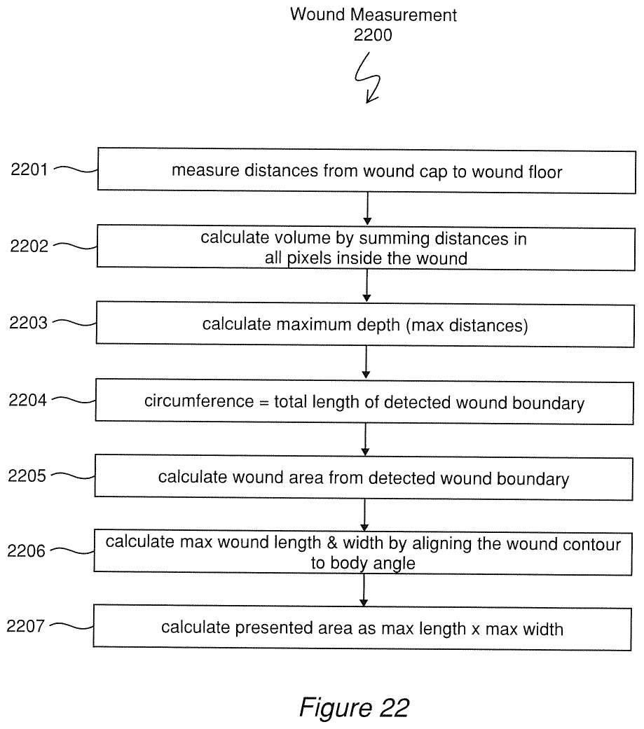

FIG. 20 presents an exemplary series of steps for the wound scan, detect, and measure block 1905 from method 1900 (FIG. 19). As with the steps in FIG. 19, the steps in FIG. 20 may be performed using exemplary interfaces introduced above. Interface 800 from FIG. 8 facilitates imaging acquisition using a 3D depth and 3D camera module (block 2001). The interface 800 provides a preview of the video images and allows a user to conveniently select a wound to measure (block 2002). The user aims at or near the center of the wound to be imaged from a predetermined proper distance (e.g., 6-18 inches) (block 2003). The user then uses the device to perform wound scan 2004, which entails starting to scan (block 2005), manipulating the camera around the wound (e.g., around the wound center) (block 2006), and then stopping the scan (block 2007). During the wound scan of block 2004 the device performs real-time wound tracking and data collection; this information may be output to another device, such as remote device, as it is being collected. The (remote or local) device may then immediately process the scan data and generate a 3D model 1001 that is displayed and available for viewing and manipulation by a user through interface 1000 (block 2008). Wound detection (block 2100) follows the wound scan and is expanded and explained below using FIG. 21. Wound measurement (block 2200) follows the wound detection and is expanded and explained below using FIG. 22.

FIG. 21 presents an exemplary series of steps for the wound detection block 2100 of FIG. 20. At block 2101, a Z-buffer is generated from the 3D wound model (the model from block 2008 of FIG. 20). Z-buffer, or depth buffer, is a term of art within computer graphics which refers to a two-dimensional array (X and Y) that stores the Z-value of each pixel. A Z-buffer may be generated with the help of an application programming interface such as Open Graphics Library (OpenGL). At block 2102 the Z-buffer is converted to a depth image. At block 2103 a region of interest (“U”) is defined for wound detection. At block 2104 wound capping is performed. Wound capping is a digital or virtual process which estimates within the three-dimensional space of the wound model the optimal upper closure (cap) for the wound. Generally such a cap corresponds with where natural skin existed prior to the patient developing the wound. For this reason, wound capping is sometimes characterized as “reconstructing” a skin surface over the wound. At block 2105 automatic detection is performed of a rough preliminary wound boundary in the 3D depth image. A suitable algorithm for acquiring a preliminary wound boundary is the Chan-Vese algorithm described in Chan and L. Vese, Active contours without edges. IEEE Trans. Image Processing, 10(2):266-277, February 2001. There is a possibility that the preliminary wound boundary is below the cap surface. In this event the cap surface is raised. At block 2106 the wound boundary is refined. A preliminary wound boundary produced by the Chan-Vese algorithm or by alternative approaches known in the art may generally require refinement for improvement accuracy. Refinement may comprise, for each pixel in the preliminary wound boundary, searching for a maximum value of a directional second derivative of the depth image along a direction orthogonal to the preliminary wound boundary, and setting a pixel of the final wound boundary to coordinates corresponding with the maximum value, subject to a size control function to avoid breaking continuity of the final wound boundary. At block 2107, after both the rough and fine automated boundary detection steps are performed, the operator is allowed to manually adjust bound borders and define the wound-body orientation. In this way a doctor or other health professional is able to serve as a check on the accuracy of the automated software, and to supply a subjective assessment which is sometimes required for wounds without objectively incontestable boundaries.

In some embodiments, a rough preliminary wound boundary may be produced not by an automated algorithmic process but instead by an operator physically marking a wound contour. This may be performed using a finger or stylus and tracing what the user perceives as the boundary on the display screen of the device. This step may accompany the step of the user defining wound-body orientation.

FIG. 22 presents an exemplary series of steps for the wound measurement block 2200 of FIG. 20. Several measurements may be desired of exemplary embodiments by users. A non-limiting list of measurements which exemplary embodiments may automatically determine and then display or export for a user is volume beneath wound cap, wound circumference, width, length, maximum depth, “rectangular” area (as determined by width×length), and exact area (true geometric area; this will agree with the rectangular area only in the unlikely event a wound is precisely rectangular). In FIG. 22, block 2201 comprises measuring distances from the wound cap to wound floor. With the wound cap already determined as described above, and the floor/walls of the wound acquired through the scanning of the wound, a “closed” three-dimensional space is specified by the 3D wound model. Block 2202 comprises calculating the volume of this wound space by, for example, summing distances in all pixels inside the wound. At block 2203 the maximum depth is determined as the maximum distance from cap to floor. At block 2204 the circumference (the perimeter length) is determined as the total length of the detected wound boundary. At block 2205 the exact (geometric) area is determined from the detected wound boundary. At block 2206 wound (max) length and (max) width are determined after aligning the wound contour to the body angle, which serves as the frame of reference. At block 2207 the “rectangular” area is determined as the max length×max width.

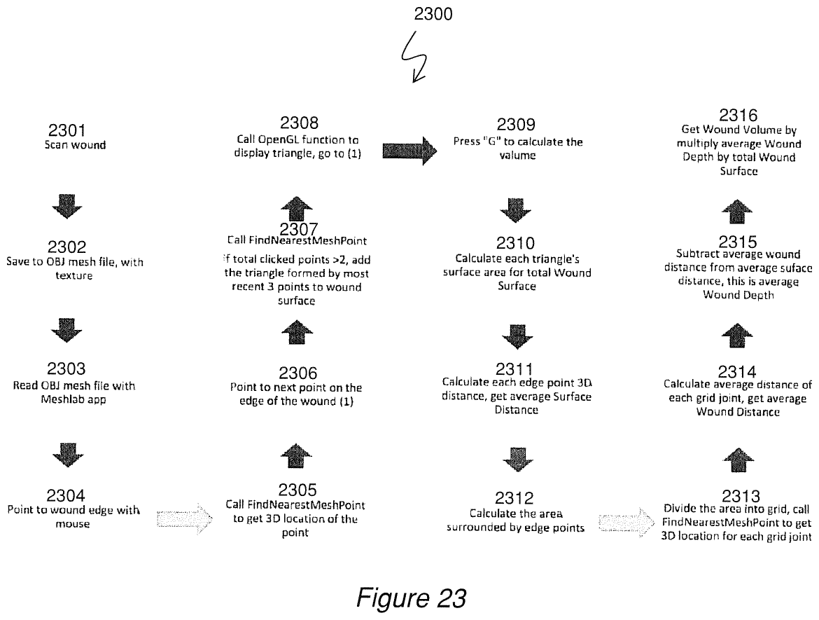

FIG. 23 shows a method 2300 for obtaining a measure of wound volume. Method 2300 may be used, for example, for block 2202 of FIG. 22. The steps as outlined in FIG. 23 are:

2301—prompting a first user to perform wound-scanning by operating a 3D camera to scan the wound and obtain a wound image, wherein the wound image has a wound edge;
2302—saving the wound image to an OBJ mesh file, with texture;
2303—reading the OBJ mesh file;
2304—prompting a second user to point to the wound edge with the mouse or pointer at a first point P1;
2305—getting 3D location of the first point P1 where the mouse was pointed;
2306—prompting the second user to point to a next point Pn on the wound edge;
2307—getting 3D location of the point Pn where the mouse was pointed, and, if a total of clicked points is greater than 2, adding the triangle formed by the most recent 3 clicked points to a wound surface value;
2308—displaying a triangle T1;
2309—pressing a predetermined key, or clicking a preset button, to calculate the wound volume;
2310—for each triangle T1 . . . Tn, calculating a surface area of the triangle for total wound surface;
2311—calculating each edge point 3D distance, thereby getting average surface distance;
2312—calculating an area surrounded by edge points;
2313—dividing the area surrounded by edge points into a grid, and getting 3D location for each grid joint;
2314—calculating average distance of each grid joint, thereby getting an average wound distance;
2315—subtracting average wound distance from average surface distance, to obtain average wound depth;
2316—obtaining wound volume by multiplying average wound depth by total wound surface.

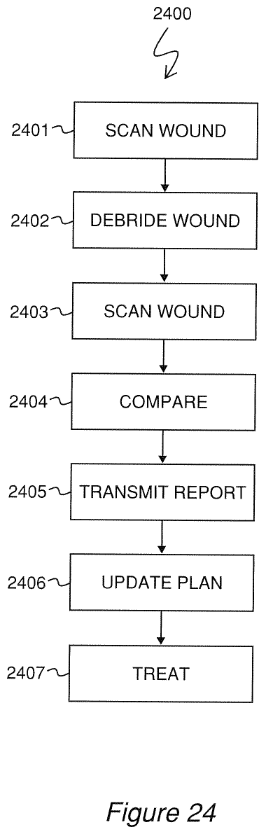

FIG. 24 shows a method 2400 for (sharp) debriding a wound and monitoring the wound through the debridement process. Sharp debridement of a wound generally entails the removal of damaged or dead tissue and/or elements like foreign objects, debris, (excess) bodily fluids, and bacteria from a wound site. A general objective of debridement is to improve the healing process and potential for the tissue that remains after debridement. Debridement is estimated to form as much as 90% of the profit in the wound care industry. Nevertheless, approximately 30-50% of medical expenses attributed or attributable to debridement are rejected by insurance carriers. Much this rejection is not because debridement is uncovered by healthcare policies, but because the documentation required to support a reimbursement claim is frequently inadequate.

Method 2400 improves the monitoring and reporting of wound progress and treatment where debridement, particularly sharp debridement, is concerned. At block 2401, using exemplary devices disclosed above, a user scan's a patients wound in need of debridement to produce a first digital record. The wound may then be debrided at block 2402 according to existing methods of debridement or methods of debridement developed in the future. Importantly, the patient's wound is scanned for a second time at block 2403 within a predetermined period of time after the debridement to produce a second digital record. Generally, it is desirable that the wound is scanned immediately after the debridement, e.g., during the same visit to a healthcare office like a doctor's office or hospital. The second scan is preferably performed within 6 hours, more preferably within 3 hours, or within 1 hour, or within 30 minutes, or within 10 minutes after the debridement is finished. The first and second digital records are then compared automatically at block 2404. The device or system automatically determines metrics for the quantitative comparison of the patient's wound as recorded in the first digital record versus in the second digital record. Exemplary metrics include but are not limited to change in area from one scan to the other, and change in wound volume from one scan to the other. Qualitative comparative measures may also be made. A report containing the results of the comparison is then transmitted at block 2405 to a user. The transmitted report may be accompanied by or else include copies of the first and second digital records. Time elapsed between the scans may also be included in the report. The user recipient of the report may be a doctor, nurse, or medical tech, for example, who may change or update a wound healing plan at block 2406 and treat the wound in question accordingly at block 2407. The user recipient of the report may be an insurance company that processes a reimbursement claim (such as but not limited to an insurance reimbursement claim) in reliance on the report as supporting documentation.

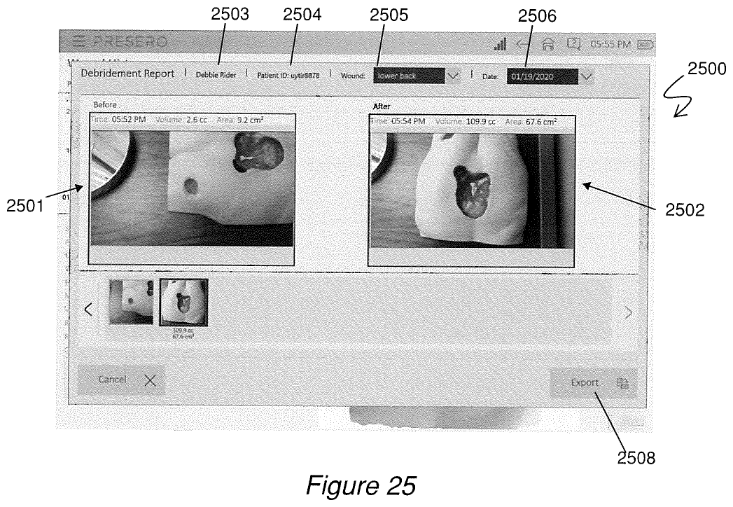

FIG. 25 shows an exemplary interface 2500 for displaying to a user the contents of the debridement report and provide options for manipulating and exporting the report. Clearly presented side-by-side are ‘before’ 2501 and ‘after’ 2502 digital records corresponding to the first (that is, pre-debridement) wound scan and the second (that is, post-debridement) wound scan. A snapshot of each digital record may be provided which includes a 2D still picture from the scan or a 2D snapshot of a 3D model produced from the scan, the time the scan was taken, the volume of the wound at the time of the scan, and the area of the wound at the time of the scan. Also included on the same screen without the user needing to click or navigate anywhere else are patient identifiers such as name 2503 and a unique alpha-numeric code 2504, a wound label 2505, and the date 2506 of the scans. While the presentation of such data may on its face appear natural to include with the report, providing a single interface 2500 that includes such data together with snapshots from both the before scan and after scan has the ability to significantly reduce human error in the reporting process for debridement and alert a user to any defects in the report prior to its export. For instance, the pictographic preview of each digital record 2501 and 2502 provides an efficient yet effective means to review the scans and give the opportunity to repeat the post-debridement scan if necessary for any reason. Once a user is satisfied with the preview of the report contents displayed in interface 2500, the user can select the export button 2508 by which the report may be automatically and seamlessly sent to predetermined recipients such as those discussed above in connection with FIG. 24.

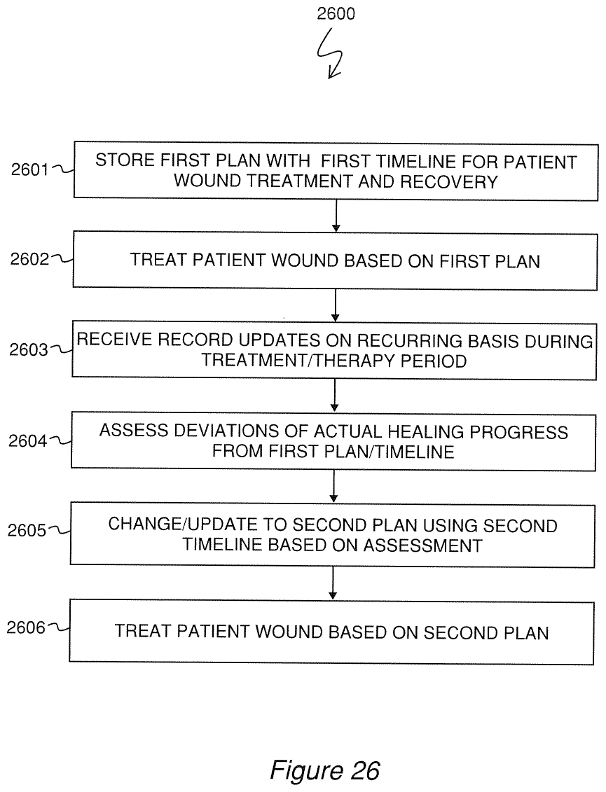

FIG. 26 is a method 2600 of patient customized wound treatment and healing predictions. At block 2601, a first wound healing plan is generated and stored for a patient's wound. The wound healing plan may include a treatment plan generated by processes described in this disclosure. A difference does exist in the wound healing plan stored at block 2601, however, and a treatment plan like the sample treatment plan 1701 shown in interface 1700 of FIG. 17. The treatment plan 1701 was customized to a present, existing state of a wound, for immediate use on that wound. Generally a treatment plan 1701 is designed for a single day's treatment of a wound, or at most the treatment of a wound for the time between visits to a healthcare professional, e.g., one or two weeks, or the time between successive changes of wound dressings. By contrast, a wound healing plan stored at block 2601 is a “big picture” plan which includes, among other data, a total time estimation from the present to the future date when the wound is expected to be fully healed. The prognosis of the wound is also taken into account to determine and reflect expected recovery rates and progress goals for the wound. For instance, wound healing progress is frequently assessed by reduction in total area of the wound from one scan to the subsequent scan. This rate may be expressed as a percentage, and each day or each week (or some other unit of time) may be assigned a particular target area reduction percentage within the wound healing plan stored at block 2601.

Block 2602 entails actual treatment of the patient wound based on the first plan from block 2601. Healthcare professionals may look up and decide upon what treatment options (e.g., what dressings, what procedures such as lavages, what medications like antibiotics or anti-inflammatories or steroids, etc. to administer) to use on the patient's wound based on the first plan from block 2601.

Blocks 2603, 2604, and 2605 represent a significant improvement on existing approaches to wound treatment. These three steps entail a semi- or fully automated update procedure for changing or replacing the first wound healing plan based on the actual progress of the patient's wound as the treatment progresses. It is worth reiterating here that wound treatment typically requires several weeks, e.g., 5 to 15 weeks to 6 months or longer, for full recovery.

Block 2603 entails receiving digital records of the patient wound on a recurring basis over a duration of time required by the patient wound to make progress healing (whether or not healing is actually taking place). The records received in each instance include wound scan data, e.g., produced according to the exemplary procedures and devices already described above. Thus in exemplary embodiments metrics such as the volume and area of the wound are rigorously and precisely tracked on a repeating (e.g., periodic) basis over the course of weeks or months. The collection of these records, which generally will number in the tens, dozens, or even hundreds, provides a detailed record of the progress (or lack thereof) of the wound. Block 2604 entails using these records to assess deviations of the actual healing progress from the first plan produced and stored back at block 2601. Traditionally the lack of detailed historic data of wound progress meant either no comprehensive wound healing plan or only a barebones wound healing plan was possible, and such a plan may have been implemented without regard for a patient's unique wound or without regard to the actual progress the patient's wound was or was not making. For instance, every patient at a particular healthcare facility with a burn wound may have been subject to a 10 week recovery plan with the same milestones for every patient, for every wound. The 10 week plan may have been immutable, even if say at week 5 the patient's wound was healing at a rate that might suggest the need for adjustment to a 20 week plan. By contrast, the assessment at block 2604 serves to determine whether actual healing progress of a wound sufficiently corresponds with projected or estimated healing progress (e.g., as quantified by wound size, volume, and/or area) based on the first plan from block 2601. When the assessed deviation at block 2604 exceeds a predetermined threshold (e.g., the patient's wound area at any given week is more than 5% off or more than 10% from the projected wound area for that week), the patient is switched to a second wound healing plan at block 2605 which is a change, update, or replacement to the first wound healing plan. The second wound healing plan includes a second timeline of treatment for the patient which differs from the first timeline. It should be appreciated that wound healing progress may be slower than anticipated or it may in fact be faster than anticipated. That is to say, in some cases a wound may actually heal faster than originally anticipated, and the second plan at block 2605 reflects a more aggressive timeline of recovery (e.g., a shorter timeline to completion) than the first plan at block 2601. The patient is then treated at block 2606 in accordance with the second plan. It should be appreciated that method 2600 may be repeated many times, and thus “first” and “second” plans may be any two successive plans within a plurality of plans produced and relied upon for treating a patient over the course of weeks or months.

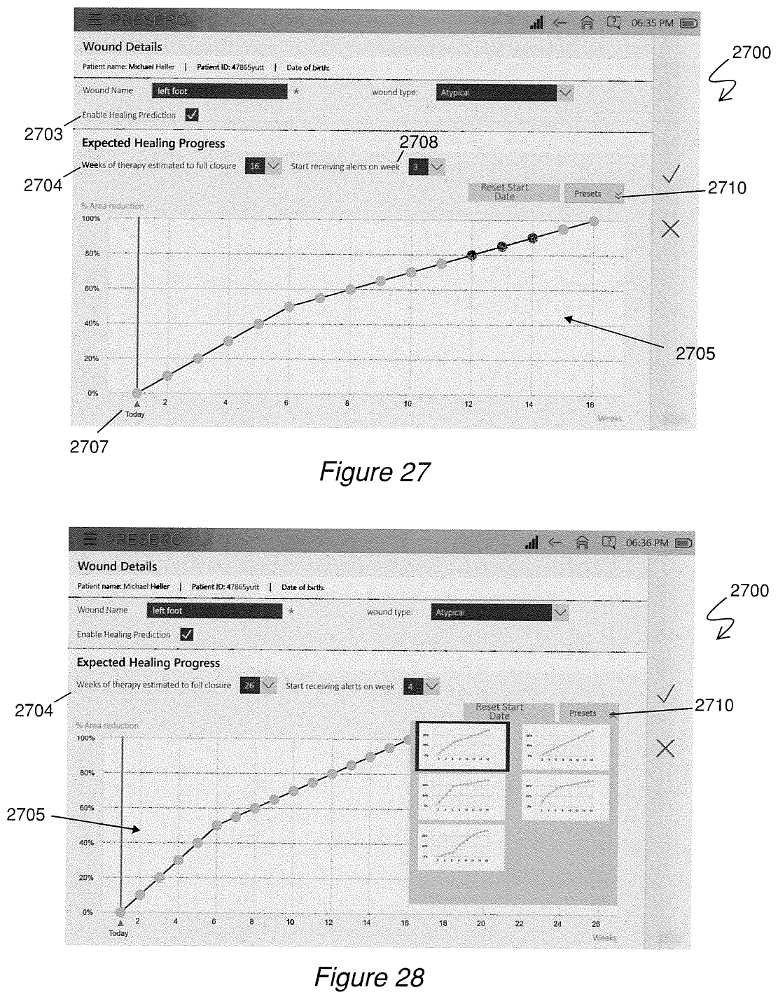

FIG. 27 provides an example interface 2700 which pictorially and/or graphically shows a user a wound healing plan. Within the interface patient and wound details are clearly presented, including in this example patient name, an alpha-numeric patient ID, date of birth, wound name, and wound time. A user may select or de-select an option 2703 to show or hide automatic healing predictions based on existing scan(s) of the patient wound. A user may manually enter or select predetermined metrics listed in the interface, including for example metric 2704, weeks of therapy estimate to full closure of the wound. A (time)line chart 2705 shows the user the total projected timeline of recover, with area reduction (by percent) as compared to the initial wound area as the metric of wound healing progress and targets based on week since the beginning of treatment. An indicator 2707 of the present day may be overlaid on the timeline to facilitate ease in assessing the timeline. Individual targets for individual weeks (generally represented by individual points on the line graph) may be adjusted manually by a user override if desired. Each data point, representing the recovery goal for a specific week in the recovery period, may be selected and dragged up or down by a user to be set at different % area reduction (or % volume reduction, or other dependent variable).

A particular issue with existing plans for tracking wound progress is a failure to identify when a patient's wound is not keeping pace with the plan, either because the wound is healing slower than originally/previously expected or because the wound is healing faster than originally/previously expected. Within interface 2700 is one or more alert setting options 2708. An example alert setting is a start date for receiving alerts. Referring back to FIG. 26, when at block 2604 deviations of actual recovery from projected recovery exceed a predetermined threshold, an alert may be automatically generated and sent to one or more interested parties informing such entities that the most recent wound healing plan may require change. Interested parties may include but are not limited to the patient and one or more users.

Wounds sometimes follow predictable phases of recovery during which metrics of recovery like rates of % area reduction or % volume reduction accelerate or decelerate for a period of time. Interface 2700 leverages this known trend in wound recovery by providing users with a list of presets 2710 shown expanded in FIG. 28. It will also be noted that FIG. 28 shows automatic adjustment of the timeline 2705 based on manual changes to the metrics 2704 allowed to the user through interface 2700. The presets in this particular example of interface 2700 include five different and distinct timelines of recovery.

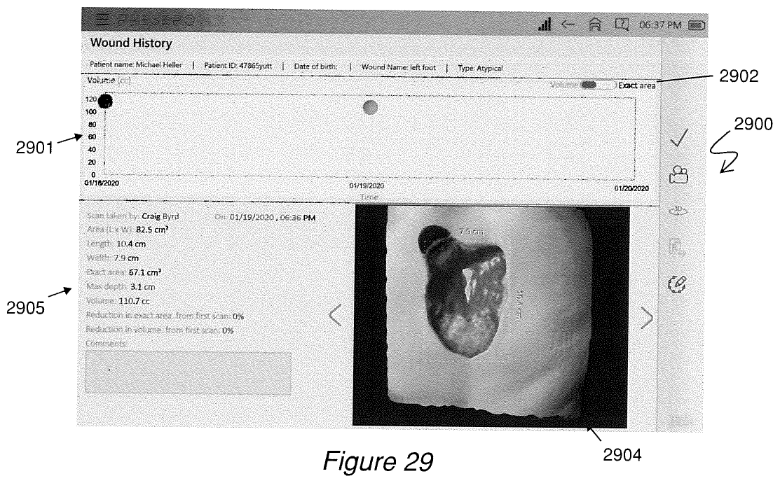

FIG. 29 shows an exemplary interface 2900 which displays and permits a user to view simultaneously a patient's actual wound healing progress, most up-to-date wound metrics 2905, and visuals 2904 of the wound (e.g., a 2D photographic image, a 3D model, etc.). The visuals 2904 may be viewable only one at a time to avoid crowding the display. Plot/chart 2901 shows a wound progress metric plotted over time. A unit toggle option 2902 allows the progress metric to be switched between volume and exact/geometric area. Some embodiments may offer additional or alternative progress metrics, but volume and exact geometric area are favored in the industry. As illustrated the figures reflect a patient wound with only two scans on record so far taken one day apart, resulting in two data points. Further points are added to chart 2901 as additional scans are made during the healing period of the wound in question. The metrics 2905 may include such quantitative measures as area (L×W), length, width, exact area, max depth, volume, reduction in area since first or most recent scan, reduction in volume since first or most recent scan, etc. Qualitative metrics may include the operator of the most recent scan, the date (and time) the scan was taken, and any comments entered by the user.

Exemplary embodiments permit evaluation of a wound, obtaining measures like wound volume, without any ruler, grid, marker, or other physical object needing to be placed on or in approximate contact with the patient. Advantageously, contact of any kind with the patient's wound or skin near the patient's wound may be avoided. “Touchless” may be used in this disclosure to mean that the patient's wound and the wound's environment is untouched by any ruler, grid, marker, 3D camera, frame enclosure holding a 3D camera, or the like.

A wound is generally defined as a break in the epithelial integrity of the skin. Such an injury, however, may be much deeper, including the dermis, subcutaneous fat, fascia, muscle, and even bone. Proper wound healing is a highly complex, dynamic, and coordinated series of steps leading to tissue repair. Acute wound healing is a dynamic process involving both resident and migratory cell populations acting in a coordinated manner within the extra-cellular matrix environment to repair the injured tissues. Some wounds fail to heal in this manner (for a variety of reasons) and may be referred to as chronic wounds.

Following tissue injury, the coordinated healing of a wound will typically involve four overlapping but well-defined phases: hemostasis, inflammation, proliferation, and remodeling. Hemostasis involves the first steps in wound response and repair that are bleeding, coagulation, and platelet and complement activation. Inflammation peaks near the end of the first day. Cell proliferation occurs over the next 7-30 days and involves the time period over which wound area measurements may be of most benefit. During this time fibroplasia, angiogenesis, re-epithelialization, and extra-cellular matrix synthesis occur. The initial collagen formation in a wound typically peaks in approximately 7 days. The wound re-epithelialization occurs in about 48 hours under optimal conditions, at which time the wound may be completely sealed. A healing wound may have 15% to 20% of full tensile strength at 3 weeks and 60% of full strength at 4 months. After the first month, a degradation and remodeling stage begins, wherein cellularity and vascularity decrease and tensile strength increases. Formation of a mature scar often requires 6 to 12 months.

The term “tissue site” may be used herein to refer to a wound or defect located on or within any tissue, including but not limited to, bone tissue, adipose tissue, muscle tissue, neuro tissue, dermal tissue, vascular tissue, connective tissue, cartilage, tendons, or ligaments. The term “tissue site” may further refer to areas of any tissue that are not necessarily wounded or defective, but are instead areas in which it is desired to add or promote the growth of additional tissue. For example, reduced pressure tissue treatment may be used in certain tissue areas to grow additional tissue that may be harvested and transplanted to another tissue location.

U.S. Pat. No. 10,593,057 (application Ser. No. 15/850,558, filed Dec. 21, 2017) is herein incorporated by reference to the extent it does not conflict with the instant disclosure.

Embodiments of the present invention may be a system, a method, and/or a computer program product. The computer program product may include a computer readable storage medium (or media) having computer readable program instructions thereon for causing a processor to carry out aspects of the present invention.

The computer readable storage medium may be a tangible device that is able to retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein may be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of embodiments may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, may be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

As will be apparent to those of skill in the art upon reading this disclosure, each of the individual embodiments described and illustrated herein has discrete components and features which may be separated from or combined with the features of any of the other several embodiments without departing from the scope or spirit of the present invention. Any recited method can be carried out in the order of events recited or in any other order which is logically possible.

While exemplary embodiments of the present invention have been disclosed herein, one skilled in the art will recognize that various changes and modifications may be made without departing from the scope of the invention as defined by the following claims.

What is claimed is:

1. A method of patient customized wound treatment, comprising
storing a digital record of a wound containing 3D wound scan data, the 3D wound scan data being produced by scanning the wound while manipulating a 3D camera around a center of the wound;
collecting responses from a user to predetermined questions pertaining to wound treatment;
automatically selecting one or more specific medical products or treatments for use treating the wound, the selection being based on the digital record and collected responses; and
transmitting a signal to one more users identifying the specific medical products or treatments.

2. The method of claim 1, further comprising steps of
scanning the wound with a 3D camera;
manipulating the 3D camera around a center of the wound during the scanning step; and
producing a 3D model of the wound from imaging data of the 3D camera, the 3D model being displayed or displayable on a screen and manipulatable on the screen to show a back or underside of the wound.

3. The method of claim 2, further comprising, during the scanning and manipulating steps, displaying to the user instructions for moving the 3D camera with respect to the wound.

4. The method of claim 1, further comprising a step of fulfilling an order of medical supplies that includes the specific medical products or treatments.

5. The method of claim 1, wherein the transmitted signal is or includes a medical reimbursement claim.

6. The method of claim 1, further comprising displaying a plurality of interfaces in series, each interface requiring the user to make at least one selection in the interface concerning a wound care topic prior to advancing to the subsequent interface.

7. The method of claim 6, wherein the plurality of interfaces includes a respective interface for each of: wound type, debridement, orientation, wound border confirmation, wound drainage, wound filler, and wound characteristics.

8. The method of claim 6, wherein each interface requires, at a minimum, a user selection from a finite list of predetermined options.

9. The method of claim 1, wherein the automatic selection step is performed with one or more of a decision tree and machine learning.

10. A computer program product for patient customized wound treatment, the computer program product comprising a computer readable storage medium having programs instructions embodied therewith, the program instructions executable by a device to cause the device to perform a method comprising:
producing a digital record of a wound containing 3D wound scan data, the 3D wound scan data being produced by scanning the wound while manipulating a 3D camera around a center of the wound;
collecting responses from a user to predetermined questions pertaining to wound treatment;
automatically selecting one or more specific medical products or treatments for use treating the wound, the selection being based on the digital record and collected responses; and
transmitting a signal to one more users identifying the specific medical products or treatments.

11. The computer program product of claim 10, wherein the program instructions further cause the device to perform
scanning the wound with a 3D camera; and
producing a 3D model of the wound from imaging data of the 3D camera, the 3D model being displayed or displayable on a screen and manipulatable on the screen to show a back or underside of the wound.

12. The computer program product of claim 11, wherein the program instructions further cause the device to perform, during the scanning step, displaying to the user instructions for moving the 3D camera with respect to the wound.

13. The computer program product of claim 10, wherein the program instructions further cause the device to perform fulfilling an order of medical supplies that includes the specific medical products or treatments.

14. The computer program product of claim 10, wherein the transmitted signal is or includes a medical reimbursement claim.

15. The computer program product of claim 10, wherein the program instructions further cause the device to perform displaying a plurality of interfaces in series, each interface requiring the medical professional to make at least one selection in the interface concerning a wound care topic prior to advancing to the subsequent interface.

16. The computer program product of claim 15, wherein the plurality of interfaces includes a respective interface for each of: wound type, debridement, orientation, wound border confirmation, wound drainage, wound filler, and wound characteristics.

17. The computer program product of claim 15, wherein each interface requires, at a minimum, a selection from a finite list of predetermined options.

18. The computer program product of claim 10, wherein the automatic selection step is performed with one or more of a decision tree and machine learning.

19. A method of monitoring wound debridement, comprising
scanning a patient's wound in need of debridement, the scan being performed with a 3D camera system to produce a first digital record;
debriding the patient's wound after the first digital record is produced;
scanning the patient's wound within a predetermined period of time after the debridement, the scan producing a second digital record;
determining metrics for quantitative comparison of the patient's wound as recorded in the first digital record versus in the second digital record; and
transmitting a report containing the first and second digital records and the quantitative comparison to a user.

20. The method of claim 19, further comprising changing or updating a wound healing plan for the patient's wound based on the transmitted report.

21. The method of claim 19, wherein the transmitted signal is or includes a medical reimbursement claim.

22. A method of patient customized wound treatment, comprising
storing a first wound healing plan that includes a first timeline of treatment for a patient wound;
receiving digital records of the patient wound on a recurring basis over a duration of time required by a patient wound to make progress healing, the digital records including wound scan data;
assessing, on the recurring basis, deviation from the first wound healing plan based on the digital records;
changing or updating the first wound healing plan to a second wound healing plan when the assessed deviation exceeds a threshold, wherein the second wound healing plan includes a second timeline of treatment for the patient wound different from the first timeline of treatment.
